(19) United States
(12) Patent Application Publication
(10) Pub. No.: US 2011/0045054 A1
(43) Pub. Date: Feb. 24, 2011

(54) MATERIALS AND METHODS FOR TREATING AND MANAGING PLAQUE DISEASE

(75) Inventors: Elazer Edelman, Brookline, MA (US); Anupam Dalal, Brookline, MA (US); Helen Marie Nugent, Needham, MA (US); Steve Bollinger, Mansfield, MA (US)

Correspondence Address: K&L Gates LLP, STATE STREET FINANCIAL CENTER, One Lincoln Street, BOSTON, MA, 02111-2950, US

(73) Assignee: PERVASIS THERAPEUTICS, INC., Cambridge, MA (US)

(21) Appl. No.: 12/880,860

(22) Filed: Sep. 13, 2010

Related U.S. Application Data
(63) Continuation of application No. 11/792,335, filed on Jun. 5, 2007, now abandoned.
(60) Provisional application No. 60/634,155, filed on Dec. 8, 2004.
(60) Provisional application No. 60/663,859, filed on Mar. 21, 2005.
(60) Provisional application No. 60/682,054, filed on May 18, 2005.

(51) Int. Cl. A61K 9/00; A61K 35/12; A61P 7/04; A61P 9/10; A61K 35/36

(52) U.S. Cl. 424/423; 424/93.7

(57) ABSTRACT

Disclosed herein are materials and methods suitable for treating and managing plaque disease, including vulnerable plaque. An implantable material comprising cells, such as but not limited to endothelial cells, and a biocompatible matrix can reduce progression or deterioration of a plaque-associated lesion situated on the interior lumen of said blood vessel. The implantable material is implanted directly on an exterior surface of a blood vessel at or adjacent or in the vicinity of the site of a lesion on an interior lumen. Alternatively, the implantable material is deposited on an exterior surface at or adjacent or in the vicinity of the site of a lesion on an interior lumen by an intraluminal delivery device which traverses or penetrates the vessel wall or by a percutaneous delivery device which enters the perivascular space. Both modes of administration can be preceded by or coincident with an imaging step. The present invention can treat hemorrhage, erosion, fissure, plaque-associated thrombosis and occlusion, rupture, displacement and/or dislodgement of a plaque lesion.

### RELATED APPLICATION DATA

This non-provisional patent application filed on Dec. 6, 2005, claims the benefit under 35 U.S.C. Section 119(e) of provisional patent application, U.S. Ser. No. 60/634,155 filed on Dec. 8, 2004; provisional patent application, U.S. Ser. No. 60/663,859 filed on Mar. 21, 2005; provisional patent application, U.S. Ser. No. 60/682,054 filed on May 19, 2005; provisional patent application, U.S. Ser .No. 60/______ filed on ______; and, claims priority under 35 U.S.C. Sections 120, 363 and/or 365 to co-pending international application PCT/US______ filed on even date herewith (also known as Attorney Docket No. ELV-002PC); and co-pending international application PCT/US_______ filed on even date herewith (also known as Attorney Docket No. ELV-009PC); the entire contents of each of the foregoing incorporated by reference herein.

### BACKGROUND OF THE INVENTION

Treatment and management of plaque disease such as vulnerable plaque disease remains an unmet clinical challenge. Onset and progression of the disease usually goes undetected until manifest in an incident of acute coronary syndrome (ACS). The risk of an episode of other more serious clinical sequelae, such as myocardial infarction, or even sudden cardiac death, becomes significantly pronounced. In spite of the prevalence and severity of plaque disease, a mode of clinical intervention pre- and post-ACS has heretofore been unavailable.

It is currently thought that plaque, both non-vulnerable and vulnerable, form from the absorption of fat droplets by the artery, causing the release of cytokines and the initiation of inflammation. Cytokines attract monocytes to the vessel wall, which infiltrate past the intima and become macrophages. The macrophages begin to soak up additional fat droplets, becoming foam cells, most likely caused by factors such as macrophage colony-stimulating factor. What started as a few fat droplets transitions into a lipid pool or necrotic core within the media of the vessel wall, with the formation of a fibrous cap at the intima.

Plaques with thick fibrous caps, plaques with little or no lipid pool, and/or eroded plaques characterized by loss or dysfunction of the luminal endothelial cells, for example, are thought to be non-vulnerable. Although the likelihood of rupture and subsequent clinical sequelae are diminished in the case of a non-vulnerable plaque, it is likely that both non-vulnerable and vulnerable plaque would benefit from treatment and management.

Further inflammation increases the size of the lipid pool or necrotic core and increases release of proteolytic enzymes, such as elastolytic cathepsins, matrix metalloproteinases, and other enzymes from macrophages, increasing the potential for rupture of the fibrous cap. Such an inflamed plaque can be referred to as a rupture-prone thin-cap fibroatheroma (TCFA). A TCFA, or any other type of rupture-prone plaque, is considered a “vulnerable,” “high-risk,” or “thrombosis-prone” plaque.

One type of vulnerable plaque can be characterized as a superficial plaque injury or a plaque erosion. Other non-ruptured vulnerable plaques can introduce an occlusive or non-occlusive thrombus extending into the lumen of the vessel, can initiate hemorrhage of the plaque and bleeding, or can initiate smooth muscle cell proliferation and/or platelet or fibrin aggregation within the plaque site. Other types of non-ruptured plaques or other forms of thrombosis in non-ruptured plaques are likely to be described in the future.

Rupture of the fibrous cap of a vulnerable plaque exposes passing blood to the lipid-rich atheromatous core, creating a high risk of thrombosis. Additionally, a plaque with an intact fibrous cap can experience leaking of the vasa vasorum and angiogenesis in the vasa vasorum, which can lead to intra-plaque hemorrhage. Such intra-plaque hemorrhages destabilize vulnerable plaques, causing plaque erosion, rupture, and acute coronary syndrome.

Furthermore, the plaque can develop a calcified nodule within the plaque site or extensive calcification within the entire circumference of the vessel, resulting in loss and/or dysfunction of endothelial cells and/or loss of the fibrous cap, creating a high-risk or vulnerable plaque.

One objective of the present invention is to provide materials and methods for treating and managing plaque disease. One such disease is vulnerable plaque.

### SUMMARY OF THE INVENTION

The present invention exploits the discovery that an intraluminal disease such as plaque disease can be treated effectively by perivascular administration of a cell-based therapy. As disclosed herein, an implantable material comprising cells, preferably endothelial cells or cells having an endothelial-like phenotype, can be used to treat and manage plaque disease when the material is situated on an exterior surface of a plaque-laden blood vessel or a blood vessel susceptible to plaque disease. This discovery permits the clinician to intervene in the development and progression of plaque disease, a disease which heretofore was not a candidate for clinical intervention or management.

According to the methods of the present invention, the implantable material can be deposited extraluminally at or adjacent or in the vicinity of the site of a plaque lesion on an interior lumen in an open-field surgical procedure. Alternatively, the implantable material can be deposited extraluminally at or adjacent or in the vicinity of the site of a lesion on an interior lumen via an intraluminal delivery device which traverses the vessel wall or a percutaneous delivery device which enters the perivascular space. It is contemplated herein that a non-luminal, also termed an extraluminal, surface can be an exterior or perivascular surface of a vessel, or can be within the adventitia, media, or intima of a blood vessel. For purposes of this invention, non-luminal or extraluminal is any surface except an interior surface of the lumen.

In one aspect, the invention provides a method of treating a plaque-burdened site on an interior lumen of a blood vessel comprising the step of contacting with an implantable material an exterior surface of said blood vessel at or adjacent or in the vicinity of a plaque-burdened site on the interior lumen of said vessel, wherein said implantable material comprises a biocompatible matrix and cells and wherein said implantable material is in an amount effective to reduce displacement or dislodgement of plaque at the plaque-burdened site; reduce plaque hemorrhage at the plaque-burdened site; reduce plaque fissure at the plaque-burdened site; reduce plaque-associated thrombosis at the plaque-burdened site; reduce plaque erosion at the plaque-burdened site; and/or reduce plaque-associated occlusion at the plaque-burdened site. Any of the modes of delivery described herein can be used to treat a plaque-burdened site.

In another currently preferred embodiment, the invention is a method of treating acute coronary syndrome comprising the step of contacting an exterior surface of a blood vessel at or adjacent or in the vicinity of a plaque-burdened site on the interior lumen of said vessel with implantable material in an amount effective to reduce the incidence of cardiac events associated with acute coronary syndrome. In yet another currently preferred embodiment, the invention provides a method of diminishing clinical sequelae associated with vulnerable plaque by contacting an exterior surface of a blood vessel at or adjacent or in the vicinity of a plaque-burdened site on the interior lumen of said vessel with implantable material in an amount effective to diminish clinical sequelae associated with vulnerable plaque. Clinical sequelae are selected from the group consisting of acute coronary syndrome, myocardial infarction, and sudden cardiac death. In other embodiments, the present invention provides a method for treating and managing plaque disease generally, preferably plaque disease associated with atherosclerosis.

In certain embodiments of the aforementioned methods, the contacting step is accomplished by first traversing an interior wall of said blood vessel and then depositing implantable material on an exterior surface of said blood vessel at or adjacent or in the vicinity of the plaque-burdened site. The traversing step is accomplished using any endovascular or intraluminal delivery device which can traverse or penetrate a blood vessel wall. In certain other embodiments, the contacting step is accomplished by directly implanting implantable material in an open-field surgical procedure. In yet other embodiments, implantable material is deposited extraluminally using a percutaneous delivery device that enters the perivascular space. For purposes of the present invention, it is contemplated that an exterior surface of a blood vessel is a non-luminal or extraluminal surface as well as a surface that occupies perivascular space. It is contemplated herein that a non-luminal, also termed an extraluminal, surface can be an exterior or perivascular surface of a vessel, or can be within the adventitia, media, or intima of a blood vessel. For purposes of this invention, non-luminal or extraluminal is any surface except an interior surface of the lumen.

With respect to any of the foregoing methods, an additional identifying step can be performed to aid in identifying a suitable implantation site. Although not required to practice the present invention, this optional step can be carried out in conjunction with either of the intraluminal or percutaneous delivery methods. This additional step can occur prior to or coincident with the intraluminal traversing step or the percutaneous entering step used to administer a flowable composition of the present invention. It can also be carried out in conjunction with any open field surgery for implanting directly either a flexible planar embodiment or a flowable composition embodiment of implantable material elsewhere as disclosed herein. It is contemplated that this identifying step can be accomplished by any suitable imaging technology, for example.

In another aspect, the invention provides an implantable material comprising a biocompatible matrix and cells suitable for use with any one of the foregoing methods. In a particularly preferred embodiment, the cells are endothelial cells. In certain currently preferred embodiments, endothelial cells are vascular endothelial cells. In yet other preferred embodiments, the cells are cells having an endothelial-like phenotype.

As contemplated and described herein, implantable material can be a flexible planar material or a flowable composition. In certain preferred embodiments, the flowable composition can be used with an intraluminal or percutaneous delivery device. The skilled clinician will appreciate the advantages presented by these various configurations of implantable material and the clinical suitability thereof.

### BRIEF DESCRIPTION OF THE DRAWINGS

FIGS. 1A and 1B are representative cell growth curves according to an illustrative embodiment of the invention.

### DETAILED DESCRIPTION OF THE INVENTION

As explained herein, the invention is based on the discovery that a cell-based therapy can be used to treat, ameliorate, manage and/or reduce the progression of plaque disease, particularly vulnerable plaque disease. The teachings presented below provide sufficient guidance to make and use the materials and methods of the present invention, and further provide sufficient guidance to identify suitable criteria and subjects for testing, measuring, and monitoring the performance of the materials and methods of the present invention.

### Plaque Disease

Identification and Monitoring of a Vulnerable Patient: A vulnerable patient is a patient with a high likelihood of developing plaque disease or coronary artery disease. A vulnerable patient can be identified, and the status of the vulnerable patient can be assessed by monitoring various biomarkers associated with plaque disease and acute coronary syndrome, for example, vulnerable plaque, vulnerable blood, and vulnerable myocardium, using routine techniques.

Identification and Monitoring of Vulnerable Plaque: Vulnerable and non-vulnerable plaque can form from absorption of fat droplets by the blood vessel. Cytokines are then released resulting in inflammation which can culminate in formation of a necrotic core within the media of the blood vessel wall and a fibrous cap at the intima. If such a fibrous cap is thick or not associated with a lipid pool, then the lesion is considered non-vulnerable and unlikely to rupture. However, if the lipid pool or necrotic core increases in size and the cap thins, it is likely to become inflamed and/or rupture-prone. Such rupture-prone lesions are considered vulnerable. Even if a lesion appears intact, intra-plaque hemorrhage can occur and ultimately destabilize the plaque followed by plaque erosion, rupture and possible ACS. Clinical manifestations of plaque disease generally, and vulnerable or non-vulnerable plaque disease specifically, are described in detail in the existing clinical literature and are well appreciated by the skilled practitioner.

In short, a vulnerable plaque can be identified and assessed according to certain clinically-significant criteria. These criteria include active inflammation, a thin fibrous cap with a large lipid pool or necrotic core, endothelial denudation with superficial platelet aggregation, fissured or injured plaque, and severe stenosis. The presence, location, and status of a vulnerable plaque can be determined by numerous methods currently know in the art. For example, a vulnerable plaque can be detected and monitored by measuring the level of C-reactive protein in a patient's blood sample, by using a baseline electrocardiogram (EKG), an exercise thallium test (a nuclear stress test), an echocardiograph, coronary angiography, or angioscopy. Moreover, additional minor criteria that can be monitored using routine methods include superficial calcium nodules, yellow color on angioscopy, intraplaque hemorrhage, endothelial dysfunction, and expansive (positive) remodeling.

Various methods of tomography can also be used to detect and monitor the status of a vulnerable plaque, including positron emission tomography (PET) scanning, optical coherence or diffuse optical tomography, fluorodeoxyglucose positron emission tomography, and other types of fluorescence-mediated tomography to detect the presence and concentration of fluorochromes in deep tissue.

Additional detection and monitoring methods include virtual histology, elastography, palpography, transcatheter colorimetry, thermography, intravascular ultrasound, intravascular magnetic resonance imaging (MRI), contrast enhanced MRI, tissue Doppler methods, electron-beam CT, multisection spiral CT, Raman spectroscopy, near-infrared (NIR) spectroscopy of protease activity induced by macrophages, or a chemometric probe that measures the acidity of portions of a blood vessel.

Identification and Monitoring of Vulnerable Blood: Vulnerable, or thrombogenic, blood is blood that contains serum markers that indicate the presence and/or status of acute cardiovascular complications, including plaque disease and coronary artery disease. Such serum markers include, but are not limited to, C-reactive protein, interleukin-6, soluble CD40 ligand, soluble intracellular adhesion molecule, circulating bacterial endotoxin, soluble human heat-shock protein 60, antibodies to mycobacterial heat-shock protein 65, and pregnancy-associated plasma protein A (PAPP-A). Other serological markers can include lipoprotein profiles, nonspecific markers of inflammation, markers of metabolic syndrome, markers of immune activation, markers of lipid peroxidation, homocysteine, circulating apoptosis markers, ADMA/DDAH, and circulating nonesterified fatty acids.

Additional serum markers associated with hypercoagulability of blood can indicate the presence and/or status of coronary artery disease. Such serum markers include, but are not limited to, fibrinogen, D-dimer, factor V Leiden, markers of increased platelet activation and aggregation, increased coagulation factors, decreased anticoagulation factors, decreased endogenous fibrinolysis activity, prothrombin mutation, other thrombogenic factors such as anticardiolipin antibodies, thrombocytosis, sickle cell disease, polycythemia, diabetes mellitus, hypercholesterolemia and hyperhomocysteinemia, increased viscosity, and transient hypercoagulability caused, for example, by smoking, dehydration, infection, adrenergic surge, cocaine, and estrogens.

Identification and Monitoring of Vulnerable Myocardium: Vulnerable myocardium is myocardium of a subject who is susceptible to acute ischemia based on the subject's autonomic nervous tone. Sympathetic hyperactivity favors the genesis of life-threatening ventricular tachyarrhythmias, whereas vagal activation exerts an antifibrillatory effect. Strong afferent stimuli from the ischemic myocardium can impair the arterial baroreflex and lead to hemodynamic instability. Factors indicating vulnerable myocardium include, but are not limited to, any type of previous atherosclerosis-related myocardial injury, such as ischemia, an old or new myocardial infarction, inflammation, fibrosis, various forms of cardiomyopathy valvular heart disease such as aortic stenosis and primary electrical disturbances, and/or commotio cordis from chest trauma.

Additional vulnerable vascular conditions which are susceptible to treatment with the present invention include any ischemic, hypoxic or injured vasculature where the vulnerable vasculature contributes to an inadequate blood supply relative to demand. Vulnerable vascular conditions can result from any injury or repair that negatively impacts blood supply. Exemplary vulnerabilities include unstable arterial syndromes such as unstable angina in the heart including a spectrum of instabilities ranging from exercise-induced angina to angina at rest; ischemia, aortic ischemia and peripheral ischemias including a spectrum of conditions ranging from intermittent caludication to gangrene, bowel ischemia in the gut, and renal ischemia, to name but a few.

### Implantable Material

General Considerations: Implantable material of the present invention comprises cells engrafted on, in and/or within a biocompatible matrix. Engrafted means securedly attached via cell to cell and/or cell to matrix interactions such that the cells withstand the rigors of the preparatory manipulations disclosed herein. As explained elsewhere herein, an operative embodiment of implantable material comprises a near-confluent, confluent or post-confluent cell population having a preferred phenotype. It is understood that embodiments of implantable material likely shed cells during preparatory manipulations and/or that certain cells are not as securedly attached as are other cells. All that is required is that implantable material comprise cells that meet the functional or phenotypical criteria set forth herein.

The implantable material of the present invention was developed on the principals of tissue engineering and represents a novel approach to addressing the above-described clinical needs. The implantable material of the present invention is unique in that the viable cells engrafted on, in and/or within the biocompatible matrix are able to supply to the vasculature multiple cell-based products in physiological proportions under physiological feed-back control. As described elsewhere herein, the cells suitable for use with the implantable material are endothelial or endothelial-like cells. Local delivery of multiple compounds by these cells and a physiologically-dynamic dosing provide more effective regulation of the processes responsible for maintaining a functional vascular structure and diminishing plaque disease. Importantly, the endothelial cells, for example, in the implantable material of the present invention are protected from the erosive blood flow within the interior vessel lumen because of its preferred placement at an extraluminal or a non-luminal surface of the vessel, for example, at the adventitia; or, contacting an exterior surface of a vessel. The implantable material of the present invention, when wrapped, deposited or otherwise contacted with such an extraluminal or non-luminal or exterior target site serves to reestablish homeostasis. That is, the implantable material of the present invention can provide an environment which mimics supportive physiology and is conducive to treat or manage plaque disease.

For purposes of the present invention, contacting means directly or indirectly interacting with an extraluminal or non-luminal surface as defined elsewhere herein. In the case of certain preferred embodiments, actual physical contact is not required for effectiveness. In other embodiments, actual physical contact is preferred. All that is required to practice the present invention is extraluminal or non-luminal deposition of an implantable material at, adjacent or in the vicinity of an injured or diseased site in an amount effective to treat the injured or diseased site. In the case of certain diseases or injuries, a diseased or injured site can clinically manifest on an interior lumen surface. In the case of other diseases or injuries, a diseased or injured site can clinically manifest on an extraluminal or non-luminal surface. In some diseases or injuries, a diseased or injured site can clinically manifest on both an interior lumen surface and an extraluminal or non-luminal surface. The present invention is effective to treat any of the foregoing clinical manifestations.

For example, endothelial cells can release a wide variety of agents that in combination can inhibit or mitigate adverse physiological events associated with acute complications associated with plaque disease. As exemplified herein, a composition and method of use that recapitulates normal physiology and dosing is useful to treat and manage plaque disease. Typically, treatment includes placing the implantable material of the present invention at, adjacent to or in the vicinity of the vulnerable vasculature, for example, in the perivascular space external to the lumen of the plaque-burdened site. When wrapped, wrapped around, deposited, or otherwise contacting an injured, traumatized or diseased blood vessel, the cells of the implantable material can provide growth regulatory compounds to the vasculature, for example to the underlying smooth muscle cells within the blood vessel. It is contemplated that, while outside the blood vessel lumen, the implantable material of the present invention comprising a biocompatible matrix or particle with engrafted cells provides a continuous supply of multiple regulatory compounds from the cells while being protected from the mechanical effects of blood flow within the interior lumen of vessel(s).

Cell Source: As described herein, the implantable material of the present invention comprises cells. Cells can be allogeneic, xenogeneic or autologous. In certain embodiments, a source of living cells can be derived from a suitable donor. In certain other embodiments, a source of cells can be derived from a cadaver or from a cell bank.

In one currently preferred embodiment, cells are endothelial cells. In a particularly preferred embodiment, such endothelial cells are obtained from vascular tissue, preferably but not limited to arterial tissue. As exemplified below, one type of vascular endothelial cell suitable for use is an aortic endothelial cell. Another type of vascular endothelial cell suitable for use is umbilical cord vein endothelial cells. And, another type of vascular endothelial cell suitable for use is coronary artery endothelial cells. Yet other types of vascular endothelial cells suitable for use with the present invention include pulmonary artery endothelial cells and iliac artery endothelial cells.

In another currently preferred embodiment, suitable endothelial cells can be obtained from non-vascular tissue. Non-vascular tissue can be derived from any tubular anatomical structure or can be derived from any non-vascular tissue or organ. Tubular anatomical structures include structures of the vascular system, the reproductive system, the genitourinary system, the gastrointestinal system, the pulmonary system, the respiratory system and the ventricular system of the brain and spinal cord.

As contemplated herein, tubular anatomical structures are those having an interior luminal surface and an extraluminal surface. For purposes of the present invention, an extraluminal or non-luminal surface can be but is not limited to an exterior surface of a tubular structure. In certain structures, the interior luminal surface is an endothelial cell layer; in certain other structures, the interior luminal surface is a non-endothelial cell layer.

In yet another embodiment, endothelial cells can be derived from endothelial progenitor cells or stem cells. In still another embodiment, endothelial cells can be derived from progenitor cells or stem cells generally. In other preferred embodiments, cells can be non-endothelial cells that are allogeneic, xenogeneic or autologous derived from vascular or non-vascular tissue or organ. The present invention also contemplates any of the foregoing which are genetically altered, modified or engineered.

In a further embodiment, two or more types of cells are co-cultured to prepare the present composition. For example, a first cell can be introduced into the biocompatible implantable material and cultured until confluent. The first cell type can include, for example, smooth muscle cells, fibroblasts, stem cells, endothelial progenitor cells, a combination of smooth muscle cells and fibroblasts, any other desired cell type or a combination of desired cell types suitable to create an environment conducive to endothelial cell growth. Once the first cell type has reached confluence, a second cell type is seeded on top of the first confluent cell type in, on or within the biocompatible matrix and cultured until both the first cell type and second cell type have reached confluence. The second cell type may include, for example, endothelial cells or any other desired cell type or combination of cell types. It is contemplated that the first and second cell types can be introduced step wise, or as a single mixture. It is also contemplated that cell density can be modified to alter the ratio of smooth muscle cells to endothelial cells.

To prevent over-proliferation of smooth muscle cells or another cell type prone to excessive proliferation, the culture procedure can be modified. For example, following confluence of the first cell type, the culture can be coated with an attachment factor suitable for the second cell type prior to introduction of the second cell type. Exemplary attachment factors include coating the culture with gelatin to improve attachment of endothelial cells. According to another embodiment, heparin can be added to the culture media during culture of the second cell type to reduce the proliferation of the first cell type and to optimize the desired first cell type to second cell type ratio. For example, after an initial growth of smooth muscle cells, heparin can be administered to control smooth muscle cell growth to achieve a greater ratio of endothelial cells to smooth muscle cells.

In a preferred embodiment, a co-culture is created by first seeding a biocompatible implantable material with smooth muscle cells to create vessel structures. Once the smooth muscle cells have reached confluence, endothelial cells are seeded on top of the cultured smooth muscle cells on the implantable material to create a simulated blood vessel. This embodiment can be administered, for example, to an AV graft or peripheral bypass graft according to methods described herein to promote the integration of the prosthetic graft material.

All that is required of the cells of the present composition is that they exhibit one or more preferred phenotypes or functional properties. As described earlier herein, the present invention is based on the discovery that a cell having a readily identifiable phenotype when associated with a preferred matrix (described elsewhere herein) can facilitate, restore and/or otherwise modulate vascular endothelial cell physiology and/or luminal homeostasis associated with the treatment of plaque disease generally.

For purposes of the present invention, one such preferred, readily identifiable phenotype typical of cells of the present invention is an ability to inhibit or otherwise interfere with vascular smooth muscle cell proliferation as measured by the in vitro assays described below. This is referred to herein as the inhibitory phenotype.

Another readily identifiable phenotype exhibited by cells of the present composition is that they are anti-thrombotic or are able to inhibit platelet adhesion and aggregation. Anti-thrombotic activity can be determined using an in vitro heparan sulfate assay and/or an in vitro platelet aggregation assay, described below.

In a typical operative embodiment of the present invention, cells need not exhibit more than one of the foregoing phenotypes. In certain embodiments, cells can exhibit more than one of the foregoing phenotypes.

While the foregoing phenotypes each typify a functional endothelial cell, such as but not limited to a vascular endothelial cell, a non-endothelial cell exhibiting such a phenotype(s) is considered endothelial-like for purposes of the present invention and thus suitable for use with the present invention. Cells that are endothelial-like are also referred to herein as functional analogs of endothelial cells; or functional mimics of endothelial cells. Thus, by way of example only, cells suitable for use with the materials and methods disclosed herein also include stem cells or progenitor cells that give rise, to endothelial-like cells; cells that are non-endothelial cells in origin yet perform functionally like an endothelial cell using the parameters set forth herein; cells of any origin which are engineered or otherwise modified to have endothelial-like functionality using the parameters set forth herein.

Typically, cells of the present invention exhibit one or more of the aforementioned phenotypes when present in confluent, near confluent or post-confluent populations and associated with a preferred biocompatible matrix such as those described elsewhere herein. As will be appreciated by one of ordinary skill in the art, confluent, near confluent or post-confluent populations of cells are identifiable readily by a variety of techniques, the most common and widely-accepted of which is direct microscopic examination. Others include evaluation of cell number per surface area using standard cell counting techniques such as but not limited to a hemacytometer or coulter counter.

Additionally, for purposes of the present invention, endothelial-like cells include but are not limited to cells which emulate or mimic functionally and phenotypcially confluent, near confluent or post-confluent endothelial cells as measured by the parameters set forth herein.

Thus, using the detailed description and guidance set forth below, the practitioner of ordinary skill in the art will appreciate how to make, use, test and identify operative embodiments of the implantable material disclosed herein. That is, the teachings provided herein disclose all that is necessary to make and use the present invention's implantable materials. And further, the teachings provided herein disclose all that is necessary to identify, make and use operatively equivalent cell-containing compositions. At bottom, all that is required is that equivalent cell-containing compositions are effective to treat, manage, modulate or ameliorate plaque disease (and all its clinical manifestations) in accordance with the methods disclosed herein. As will be appreciated by the skilled practitioner, equivalent embodiments of the present composition can be identified using only routine experimentation together with the teachings provided herein.

In certain preferred embodiments, endothelial cells used in the implantable material of the present invention are isolated from the aorta of human cadaver donors. Each lot of cells is derived from a single donor or from multiple donors, tested extensively for endothelial cell purity, biological function, the presence of bacteria, fungi, known human pathogens and other adventitious agents. The cells are cryopreserved and banked using well-known techniques for later expansion in culture for subsequent formulation in biocompatible implantable materials.

Cell Preparation: As stated above, suitable cells can be obtained from a variety of tissue types and cell types. In certain preferred embodiments, human aortic endothelial cells used in the implantable material are isolated from the aorta of cadaver donors. In other embodiments, porcine aortic endothelial cells (Cell Applications, San Diego, Calif.) are isolated from normal porcine aorta by a similar procedure used to isolate human aortic endothelial cells. Each lot of cells can be derived from a single donor or from multiple donors, tested extensively for endothelial cell viability, purity, biological function, the presence of mycoplasma, bacteria, fungi, yeast, known human pathogens and other adventitious agents. The cells are further expanded, characterized and cryopreserved to form a working cell bank at the third to sixth passage using well-known techniques for later expansion in culture and for subsequent formulation in biocompatible implantable material.

The human or porcine aortic endothelial cells are prepared in T-75 flasks pre-treated by the addition of approximately 15 ml of endothelial cell growth media per flask. Human aortic endothelial cells are prepared in Endothelial Growth Media (EGM-2, Cambrex Biosciences, East Rutherford, N.J.). EGM-2 consists of Endothelial Cell Basal Media (EBM-2, Cambrex Biosciences) supplemented with EGM-2 singlequots, which contain 2% FBS. Porcine cells are prepared in EBM-2 supplemented with 5% FBS and 50 μg/ml gentamicin. The flasks are placed in an incubator maintained at approximately 37° C. and 5% CO₂/95% air, 90% humidity for a minimum of 30 minutes. One or two vials of the cells are removed from the −160° C. to −140° C. freezer and thawed at approximately 37° C. Each vial of thawed cells is seeded into two T-75 flasks at a density of approximately 3×10³ cells per cm³, preferably, but no less than 1.0×10³ and no more than 7.0×10³; and the flasks containing the cells are returned to the incubator. After about 8-24 hours, the spent media is removed and replaced with fresh media. The media is changed every two to three days, thereafter, until the cells reach approximately 85-100% confluence preferably, but no less than 60% and no more than 100%. When the implantable material is intended for clinical application, only antibiotic-free media is used in the post-thaw culture of human aortic endothelial cells and manufacture of the implantable material of the present invention.

The endothelial cell growth media is then removed, and the monolayer of cells is rinsed with 10 ml of HEPES buffered saline (HEPES). The HEPES is removed, and 2 ml of trypsin is added to detach the cells from the surface of the T-75 flask. Once detachment has occurred, 3 ml of trypsin neutralizing solution (TNS) is added to stop the enzymatic reaction. An additional 5 ml of HEPES is added, and the cells are enumerated using a hemocytometer. The cell suspension is centrifuged and adjusted to a density of, in the case of human cells, approximately 1.75×10⁶ cells/ml using EGM-2 without antibiotics, or in the case of porcine cells, approximately 1.50×10⁶ cells/ml using EBM-2 supplemented with 5% FBS and 50 μg/ml gentamicin.

Biocompatible Matrix: According to the present invention, the implantable material comprises a biocompatible matrix. The matrix is permissive for cell growth and attachment to, on or within the matrix. The matrix is flexible and conformable. The matrix can be a solid, a semi-solid or flowable porous composition. For purposes of the present invention, flowable composition means a composition susceptible to administration using an injection or injection-type delivery device such as, but not limited to, a needle, a syringe or a catheter. Other delivery devices which employ extrusion, ejection or expulsion are also contemplated herein. Porous matrices are preferred. A preferred flowable composition is shape-retaining. The matrix also can be in the form of a flexible planar form. The matrix also can be in the form of a gel, a foam, a suspension, a particle, a microcarrier, a microcapsule, or a fibrous structure. A currently preferred matrix has a particulate form.

The matrix, when implanted on an extraluminal or non-luminal or exterior surface of a blood vessel for example, can reside at the implantation site for at least about 7-90 days, preferably about at least 7-14 days, more preferably about at least 14-28 days, most preferably about at least 28-90 days before it bioerodes.

One preferred matrix is Gelfoam® (Pfizer, Inc., New York, N.Y.), an absorbable gelatin sponge (hereinafter “Gelfoam matrix”). Gelfoam matrix is a porous and flexible surgical sponge prepared from a specially treated, purified porcine dermal gelatin solution.

According to another embodiment, the biocompatible matrix material can be a modified matrix material. Modifications to the matrix material can be selected to optimize and/or to control function of the cells, including the cells' phenotype (e.g., the inhibitory phenotype) as described above, when the cells are associated with the matrix. According to one embodiment, modifications to the matrix material include coating the matrix with attachment factors or adhesion peptides that enhance the ability of the cells to inhibit smooth muscle cell proliferation, to decrease inflammation, to increase heparan sulfate production, to increase prostacyclin production, and/or to increase TGF-β₁ production. Exemplary attachment factors include, for example, fibronectin, fibrin gel, and covalently attached cell adhesion ligands (including RGD) utilizing standard aqueous carbodiimide chemistry. Additional cell adhesion ligands include peptides having cell adhesion recognition sequences, including but not limited to: RGDY, REDVY, GRGDF, GPDSGR, GRGDY and REDV.

According to another embodiment, the matrix is a matrix other than Gelfoam. Additional exemplary matrix materials include, for example, fibrin gel, alginate, polystyrene sodium sulfonate microcarriers, collagen coated dextran microcarriers, PLA/PGA and pHEMA/MMA copolymers (with polymer ratios ranging from 1-100% for each copolymer). According to a preferred embodiment, these additional matrices are modified to include attachment factors or adhesion peptides, as recited and described above. Exemplary attachment factors include, for example, gelatin, collagen, fibronectin, fibrin gel, and covalently attached cell adhesion ligands (including for example RGD) utilizing standard aqueous carbodiimide chemistry. Additional cell adhesion ligands include peptides having cell adhesion recognition sequences, including but not limited to: RGDY, REDVY, GRGDF, GPDSGR, GRGDY and REDV.

Embodiments of Implantable Materials: As stated earlier, implantable material of the present invention can be a flexible planar form or a flowable composition. When in a flexible planar form, it can assume a variety of shapes and sizes, preferably a shape and size which conforms to a contoured exterior surface of a vessel or tubular structure when situated at or adjacent to or in the vicinity of a disease site. Examples of preferred configurations suitable for use in this manner are disclosed in co-pending application PCT/US_______ filed on even date herewith (also known as Attorney Docket No. ELV-002PC), the entire contents of which are herein incorporated by reference.

Flowable Composition: In certain embodiments contemplated herein, the implantable material of the present invention is a flowable composition comprising a particulate biocompatible matrix which can be in the form of a gel, a foam, a suspension, a particle, a microcarrier, a microcapsule, or other flowable material. The current invention contemplates any flowable composition that can be administered with an injection-type delivery device as earlier described. For example, an endovascular delivery device that can navigate the interior length of a blood vessel, or an injection-type delivery device, is suitable for this purpose as described below. The flowable composition is preferably a shape-retaining composition. Thus, an implantable material comprising cells in, on or within a flowable-type particulate matrix as contemplated herein can be formulated for use with any endovascular or injectable delivery device ranging in internal diameter from about 22 gauge to about 26 gauge and capable of delivering about 50 mg of flowable composition comprising particulate material containing preferably about 1 million cells in about 1 to about 3 ml of flowable composition.

According to a currently preferred embodiment, the flowable composition comprises a biocompatible particulate matrix such as Gelfoam® particles, Gelfoam® powder, or pulverized Gelfoam® (Pfizer Inc., New York, N.Y.) (hereinafter “Gelfoam particles”), a product derived from porcine dermal gelatin. According to another embodiment, the particulate matrix is Cytodex-3 (Amersham Biosciences, Piscataway, N.J.) microcarriers, comprised of denatured collagen coupled to a matrix of cross-linked dextran. Related flowable compositions suitable for use to treat, manage and/or ameliorate the development and/or progression of plaque disease in accordance with the present invention are disclosed in co-pending application PCT/US______ filed on even date herewith (also known as Attorney Docket No. ELV-009PC), the entire contents of which are herein incorporated by reference.

According to alternative embodiments, matrices comprising particulate materials can be modified as described above using materials and methods well known in the art.

Preparation of Implantable Material: Prior to cell seeding, the biocompatible matrix is re-hydrated by the addition of EGM-2 without antibiotics at approximately 37° C. and 5% CO₂/95% air for 12 to 24 hours. The implantable material is then removed from their re-hydration containers and placed in individual tissue culture dishes. The biocompatible matrix is seeded at a preferred density of approximately 1.5-2.0×10⁵ cells (1.25-1.66×10⁵ cells/cm³ of matrix) and placed in an incubator maintained at approximately 37° C. and 5% CO₂/95% air, 90% humidity for 3-4 hours to facilitate cell attachment. The seeded matrix is then placed into individual containers (Evergreen, Los Angeles, Calif.) tubes, each fitted with a cap containing a 0.2 μm filter with EGM-2 and incubated at approximately 37° C. and 5% CO₂/95% air. The media is changed every two to three days, thereafter, until the cells have reached confluence. The cells in one preferred embodiment are preferably passage 6, but cells of fewer or more passages can be used. Further implantable material preparation protocols according to additional embodiments of the invention are disclosed in co-pending application PCT/US______ filed on (also known as Attorney Docket No. ELV-00______), the entire contents of which are herein incorporated by reference.

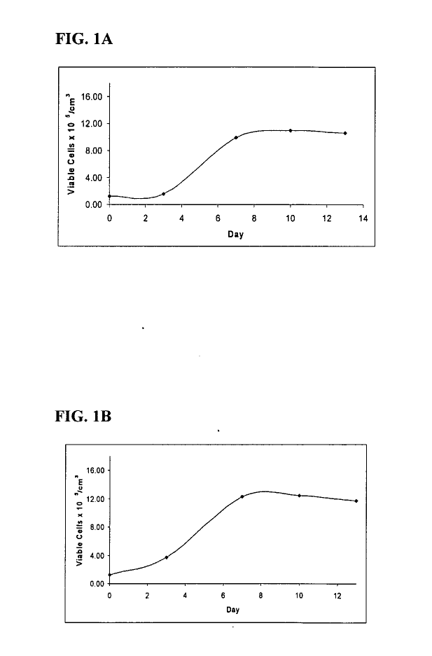

Cell Growth Curve and Confluence: A sample of implantable material is removed on or around days 3 or 4, 6 or 7, 9 or 10, and 12 or 13, the cells are counted and assessed for viability, and a growth curve is constructed and evaluated in order to assess the growth characteristics and to determine whether confluence, near confluence or post-confluence has been achieved. Representative growth curves from two preparations of implantable material comprising porcine aortic endothelial cell implanted lots are presented in FIGS. 1A and 1B. In these examples, the implantable material is in a flexible planar form. Generally, one of ordinary skill will appreciate the indicia of acceptable cell growth at early, mid- and late time points, such as observation of an increase in cell number at the early time points (when referring to FIG. 1A, between about days 2-6), followed by a near confluent phase (when referring to FIG. 1A, between about days 6-8), followed by a plateau in cell number once the cells have reached confluence (when referring to FIG. 1A, between about days 8-10) and maintenance of the cell number when the cells are post-confluent (when referring to FIG. 1A, between about days 10-14). For purposes of the present invention, cell populations which are in a plateau for at least 72 hours are preferred.

Cell counts are achieved by complete digestion of the aliquot of implantable material with a solution of 0.8 mg/ml collagenase in a trypsin-EDTA solution. After measuring the volume of the digested implantable material, a known volume of the cell suspension is diluted with 0.4% trypan blue (4:1 cells to trypan blue) and viability assessed by trypan blue exclusion. Viable, non-viable and total cells are enumerated using a hemacytometer. Growth curves are constructed by plotting the number of viable cells versus the number of days in culture. Cells are shipped and implanted after reaching confluence.

For purposes of the present invention, confluence is defined as the presence of at least about 4×10⁵ cells/cm³ when in a flexible planar form of the implantable material (1.0×4.0×0.3 cm), and preferably about 7×10⁵ to 1×10⁶ total cells per aliquot (50-70 mg) when in a flowable composition. For both, cell viability is at least about 90% preferably but no less than 80%. If the cells are not confluent by day 12 or 13, the media is changed, and incubation is continued for an additional day. This process is continued until confluence is achieved or until 14 days post-seeding. On day 14, if the cells are not confluent, the lot is discarded. If the cells are determined to be confluent after performing in-process checks, a final media change is performed. This final media change is performed using EGM-2 without phenol red and without antibiotics. Immediately following the media change, the tubes are fitted with sterile plug seal caps for shipping.

Evaluation of Functionality and Phenotype: For purposes of the invention described herein, the implantable material is further tested for indicia of functionality and their phenotype prior to implantation. For example, conditioned media are collected during the culture period to ascertain levels of heparan sulfate, transforming growth factor-β₁ (TGF-β₁), basic fibroblast growth factor (b-FGF), and nitric oxide which are produced by the cultured endothelial cells. In certain preferred embodiments, the implantable material can be used for the purposes described herein when total cell number is at least about 2, preferably at least about 4×10⁵ cells/cm³ of implantable material; percentage of viable cells is at least about 80-90%, preferably ≧90%, most preferably at least about 90%; heparan sulfate in conditioned media is at least about 0.5-1.0, preferably at least about 1.0 microg/10⁶ cell/day. TGF-β₁ in conditioned media is at least about 200-300 picog/ml/day, preferably at least about 300 picog/ml/day; b-FGF in conditioned media is below about 200 picog/ml, preferably no more than about 400 picog/ml.

Heparan sulfate levels can be quantitated using a routine dimethylmethylene blue-chondroitinase ABC digestion spectrophotometric assay. Total sulfated glycosaminoglycan (GAG) levels are determined using a dimethylmethylene blue (DMB) dye binding assay in which unknown samples are compared to a standard curve generated using known quantities of purified chondroitin sulfate diluted in collection media. Additional samples of conditioned media are mixed with chondroitinase ABC to digest chondroitin and dermatan sulfates prior to the addition of the DMB color reagent. All absorbances are determined at the maximum wavelength absorbance of the DMB dye mixed with the GAG standard, generally around 515-525 nm. The concentration of heparan sulfate per 10⁶ cells per day is calculated by subtracting the concentration of chondroitin and dermatan sulfate from the total sulfated glycosaminoglycan concentration in conditioned media samples. Chondroitinase ABC activity is confirmed by digesting a sample of purified chondroitin sulfate. Conditioned medium samples are corrected appropriately if less than 100% of the purified chondroitin sulfate is digested. Heparan sulfate levels may also be quantitated using an ELISA assay employing monoclonal antibodies.

TGF-β₁ and b-FGF levels can be quantitated using an ELISA assay employing monoclonal or polyclonal antibodies, preferably polyclonal. Control collection media can also be quantitated using an ELISA assay and the samples corrected appropriately for TGF-β₁ and b-FGF levels present in control media.

Nitric oxide (NO) levels can be quantitated using a standard Griess Reaction assay. The transient and volatile nature of nitric oxide makes it unsuitable for most detection methods. However, two stable breakdown products of nitric oxide, nitrate (NO₃) and nitrite (NO₂), can be detected using routine photometric methods. The Griess Reaction assay enzymatically converts nitrate to nitrite in the presence of nitrate reductase. Nitrite is detected colorimetrically as a colored azo dye product, absorbing visible light in the range of about 540 nm. The level of nitric oxide present in the system is determined by converting all nitrate into nitrite, determining the total concentration of nitrite in the unknown samples, and then comparing the resulting concentration of nitrite to a standard curve generated using known quantities of nitrate converted to nitrite.

The earlier-described preferred inhibitory phenotype is assessed using the quantitative heparan sulfate, TGF-β₁ and b-FGF assays described above, as well as quantitative in vitro assays of smooth muscle cell growth and inhibition of thrombosis as follows. For purposes of the present invention, implantable material is ready for implantation when one or more of these alternative in vitro assays confirm that the implantable material is exhibiting the preferred inhibitory phenotype.

To evaluate inhibition of smooth muscle cell growth in vitro, the magnitude of inhibition associated with cultured endothelial cells is determined. Porcine or human aortic smooth muscle cells are sparsely seeded in 24 well tissue culture plates in smooth muscle cell growth medium (SmGM-2, Cambrex BioScience). The cells are allowed to attach for 24 hours. The media is then replaced with smooth muscle cell basal media (SmBM) containing 0.2% FBS for 48-72 hours to growth arrest the cells. Conditioned media is prepared from post-confluent endothelial cell cultures, diluted 1:1 with 2×SMC growth media and added to the cultures. A positive control for inhibition of smooth muscle cell growth is included in each assay. After three to four days, the number of cells in each sample is enumerated using a Coulter Counter. The effect of conditioned media on smooth muscle cell proliferation is determined by comparing the number of smooth muscle cells per well immediately before the addition of conditioned media with that after three to four days of exposure to conditioned media, and to control media (standard growth media with and without the addition of growth factors). The magnitude of inhibition associated with the conditioned media samples are compared to the magnitude of inhibition associated with the positive control. According to a preferred embodiment, the implantable material is considered inhibitory if the conditioned media inhibits about 20% of what the heparin control is able to inhibit.

To evaluate inhibition of thrombosis in vitro, the level of heparan sulfate associated with the cultured endothelial cells is determined. Heparan sulfate has both anti-proliferative and anti-thrombotic properties. Using either the routine dimethylmethylene blue-chondroitinase ABC digestion spectrophotometric assay or an ELISA assay, both assays are described in detail above, the concentration of heparan sulfate per 10⁶ cells is calculated. The implantable material can be used for the purposes described herein when the heparan sulfate in the conditioned media is at least about 0.5-1.0, preferably at least about 1.0 microg/10⁶ cells/day.

Another method to evaluate inhibition of thrombosis involves determining the magnitude of inhibition of platelet aggregation in vitro associated with platelet rich-plasma. Porcine plasma is obtained by the addition of sodium citrate to porcine blood samples at room temperature. Citrated plasma is centrifuged at a gentle speed, to draw red and white blood cells into a pellet, leaving platelets suspended in the plasma. Conditioned media is prepared from post-confluent endothelial cell cultures and added to aliquots of the platelet-rich plasma. A platelet aggregating agent (agonist) is added to the plasma as control. Platelet agonists commonly include arachidonate, ADP, collagen, epinephrine, and ristocetin (available from Sigma-Aldrich Co., St. Louis, Mo.). An additional aliquot of plasma has no platelet agonist or conditioned media added, to assess for baseline spontaneous platelet aggregation. A positive control for inhibition of platelet aggregation is also included in each assay. Exemplary positive controls include aspirin, heparin, abciximab (ReoPro®, Eli Lilly, Indianapolis, Ind.), tirofiban (Aggrastat®, Merck & Co., Inc., Whitehouse Station, N.J.) or eptifibatide (Integrilin®, Millennium Pharmaceuticals, Inc., Cambridge, Mass.). The resulting platelet aggregation of all test conditions are then measured using an aggregometer. The aggregometer measures platelet aggregation by monitoring optical density. As platelets aggregate, more light can pass through the specimen. The aggregometer reports results in “platelet aggregation units,” a function of the rate at which platelets aggregate. Aggregation is assessed as maximal aggregation at 6 minutes after the addition of the agonist. The effect of conditioned media on platelet aggregation is determined by comparing baseline platelet aggregation before the addition of conditioned medium with that after exposure of platelet-rich plasma to conditioned medium, and to the positive control. Results are expressed as a percentage of the baseline. The magnitude of inhibition associated with the conditioned media samples are compared to the magnitude of inhibition associated with the positive control. According to a preferred embodiment, the implantable material is considered inhibitory if the conditioned media inhibits about 20% of what the positive control is able to inhibit.

When ready for implantation, the planar form of implantable material is supplied in final product containers, each preferably containing a 1×4×0.3 cm (1.2 cm³), sterile implantable material with preferably approximately 5-8×10⁵ or preferably at least about 4×10⁵ cells/cm³, and at least about 90% viable cells (for example, human aortic endothelial cells derived from a single cadaver donor) per cubic centimeter implantable material in approximately 45-60 ml, preferably about 50 ml, endothelial growth medium (for example, endothelial growth medium (EGM-2), containing no phenol red and no antibiotics. When porcine aortic endothelial cells are used, the growth medium is also EBM-2 containing no phenol red, but supplemented with 5% FBS and 50 μg/ml gentamicin.

In other preferred embodiments, the flowable composition (for example, a particulate form biocompatible matrix) is supplied in final product containers, including, for example, sealed tissue culture containers modified with filter caps or pre-loaded syringes, each preferably containing about 50-60 mg of flowable composition comprising about 7×10⁵ to about 1×10⁶ total endothelial cells in about 45-60 ml, preferably about 50 ml, growth medium per aliquot.

Shelf-Life of Implantable Material: The implantable material of the present invention comprising a confluent, near-confluent or post-confluent population of cells can be maintained at room temperature in a stable and viable condition for at least two weeks. Preferably, such implantable material is maintained in about 45-60 ml, more preferably about 50 ml, of transport media with or without additional FBS. Transport media comprises EGM-2 media without phenol red. FBS can be added to the volume of transport media up to about 10% FBS, or a total concentration of about 12% FBS. However, because FBS must be removed from the implantable material prior to implantation, it is preferred to limit the amount of FBS used in the transport media to reduce the length of rinse required prior to implantation.

Cryopreservation of Implantable Material: The implantable material of the present invention can be cryopreserved for storage and/or transport to the implantation site without diminishing its clinical potency or integrity upon eventual thaw. Preferably, implantable material is cryopreserved in a 15 ml cryovial (Nalgene®, Nalge Nunc Int'l, Rochester, N.Y.) in a solution of about 5 ml CryoStor CS-10 solution (BioLife Solutions, Oswego, N.Y.) containing about 10% DMSO, about 2-8% Dextran and about 50-75% FBS. Cryovials are placed in a cold isopropanol water bath, transferred to an −80° C. freezer for 4 hours, and subsequently transferred to liquid nitrogen (−150° C. to −165° C.).

Cryopreserved aliquots of the implantable material are then slowly thawed at room temperature for about 15 minutes, followed by an additional approximately 15 minutes in a room temperature water bath. The material is then washed about 3 times in about 15 ml wash media. Wash media comprises EBM without phenol red and with 50 μg/ml gentamicin. The first two rinse procedures are conducted for about 5 minutes at room temperature. The final rinse procedure is conducted for about 30 minutes at 37° C. in 5% CO₂.

Following the thaw and rinse procedures, the cryopreserved material is allowed to rest for about 48 hours in about 10 ml of recovery solution. For porcine endothelial cells, the recovery solution is EBM-2 supplemented with 5% FBS and 50 μg/ml gentamicin at 37° C. in 5% CO₂; for human endothelial cells, the recovery solution is EGM-2 without antibiotics. Further post-thaw conditioning can be carried out for at least another 24 hours prior to use and/or packaging for storage or transport.

Immediately prior to implantation, the medium is decanted and the implantable material is rinsed in about 250-500 ml sterile saline (USP). The medium in the final product contains a small amount of FBS to maintain cell viability during transport to a clinical site if necessary. The FBS has been tested extensively for the presence of bacteria, fungi and other viral agents according to Title 9 CFR: Animal and Animal Products. A rinsing procedure is employed just prior to implantation, which decreases the amount of FBS transferred preferably to between 0-60 ng per implant.

The total cell load per human patient will be preferably approximately 1.6-2.6×10⁴ cells per kg body weight, but no less than about 2×10³ and no more than about 2×10⁶ cells per kg body weight.

Administration of Implantable Material: The implantable material of the present invention when in a flowable composition comprises a particulate biocompatible matrix and cells, preferably endothelial cells, more preferably vascular endothelial cells, which are about 90% viable at a preferred density of about 0.8×10⁴ cells/mg, more preferred of about 1.5×10⁴ cells/mg, most preferred of about 2×10⁴ cells/mg, and which can produce conditioned media containing heparan sulfate at least about 0.5-1.0, preferably at least about 1.0 microg/10⁶ cell/day, TGF-β₁ at at least about 200-300 picog/ml/day, preferably at least about 300 picog/ml/day, and b-FGF below about 200 picog/ml and preferably no more than about 400 picog/ml; and, display the earlier-described inhibitory phenotype.

For purposes of the present invention generally, administration of the implantable particulate material is localized to a site in the vicinity of, adjacent or at a site of plaque disease. The site of deposition of the implantable material is extraluminal. As contemplated herein, localized, extraluminal deposition can be accomplished as follows.

In a particularly preferred embodiment, the flowable composition is first administered percutaneously, entering the perivascular space and then deposited on an extraluminal site using a suitable needle, catheter or other suitable percutaneous delivery device. Alternatively, the flowable composition is delivered percutaneously using a needle, catheter or other suitable delivery device in conjunction with an identifying step to facilitate delivery to a desired extraluminal site. The identifying step can occur prior to or coincident with percutaneous delivery. The identifying step can be accomplished using intravascular ultrasound, other routine ultrasound, fluoroscopy, and/or endoscopy methodologies, to name but a few. The identifying step is optionally performed and not required to practice the methods of the present invention.

The flowable composition can also be administered intraluminally, i.e. endovascularly. For example, the composition can be delivered by any device able to be inserted within a blood vessel. In this instance, such an intraluminal delivery device is equipped with a traversing or penetrating device which traverses or penetrates the luminal wall of a blood vessel to reach a non-luminal surface of a blood vessel. The flowable composition is then deposited on a non-luminal surface of a blood vessel at, adjacent or in the vicinity of a plaque-burdened site.

It is contemplated herein that a non-luminal, also termed an extraluminal, surface can be an exterior or perivascular surface of a vessel, or can be within the adventitia, media, or intima of a blood vessel. For purposes of this invention, non-luminal or extraluminal is any surface except an interior surface of the lumen.

The traversing or penetrating devices contemplated herein can permit, for example, a single point of delivery or a plurality of delivery points arranged in a desired geometric configuration to accomplish delivery of flowable composition to a non-luminal surface of a blood vessel without disrupting a plaque-associated lesion. A plurality of delivery points can be arranged, for example, in a circle, a bulls-eye, or a linear array arrangement to name but a few. The traversing or penetrating device can also be in the form of a stent perforator, such as but not limited to, a balloon stent including a plurality of delivery points.

According to a preferred embodiment of the invention, the penetrating device is inserted via the interior luminal surface of the blood vessel either proximal or distal to the site of the plaque-associated lesion. In some clinical subjects, insertion of the penetrating device at the site of the plaque-associated lesion could disrupt or rupture the lesion. Accordingly, in such subjects, care should be taken to insert the penetrating device at a location a distance from the plaque, preferably a distance determined by the clinician governed by the specific circumstances at hand.

Preferably, flowable composition is deposited on a perivascular surface of a blood vessel, either at the site of a lesion to be treated, or adjacent to or in the vicinity of the site of a lesion. The composition can be deposited in a variety of locations relative to a plaque-associated lesion, for example, at the lesion, adjacent to the lesion, for example, upstream of the lesion, on the opposing exterior vessel surface from the lesion. According to a preferred embodiment, an adjacent site is within about 2 mm to 20 mm of the site of the plaque-associated lesion. In another preferred embodiment, a site is within about 21 mm to 40 mm; in yet another preferred embodiment, a site is within about 41 mm to 60 mm. In another preferred embodiment, a site is within about 61 mm to 100 mm. Alternatively, an adjacent site is any other clinician-determined adjacent location where the deposited composition is capable of exhibiting a desired effect on a blood vessel in the proximity of the plaque-associated lesion.

In another embodiment, the flowable composition is delivered directly to a surgically-exposed extraluminal site at, adjacent to or in the vicinity of a site of plaque disease. In this case delivery is guided and directed by direct observation of the site. Also in this case, delivery can be aided by coincident use of an identifying step as described above. Again, the identifying step is optional.

According to another embodiment of the invention, the flexible planar form of the implantable material is delivered locally to a surgically-exposed extraluminal site at, adjacent to or in the vicinity of a site of plaque disease. In one case, at least one piece of the implantable material is applied to a desired site by passing one end of the implantable material under the vessel. The ends are then wrapped around the vessel, keeping the implantable material centered. The ends overlap each other to secure the material in place. In other cases , the implantable material does not need to completely wrap around the circumference of the vessel; it need only conform to and contact an exterior surface of the vessel and be implanted in an amount effective to treat a diseased site.

### EXAMPLES

### 1. Plaque Erosion

The pigeon model known as White Carneau (Arterioscler. Thromb. Vasc. Biol. 23:535-42 (2003); J. Hered. 92:439-42 (2001); Atherosclerosis 65:29-35 (1987); Arch. Pathol. Lab. Med. 102:581-6 (1978)) will be studied to demonstrate treatment and management of plaque disease, including plaque erosion, using the composition and methods of the present invention. Spontaneous plaque-laden animals will be identified by standard techniques such as angiography, thermography, intravascular ultrasound, and/or NIS spectroscopy to measure proteolytic activity. Two groups of animals will be maintained similarly, except one group will receive an effective amount of implantable material. Reduction of and/or amelioration of plaque disease, including plaque erosion, will be monitored over time. It is expected that pigeons treated with the materials and methods of the present invention will display reduction and/or amelioration of at least plaque erosion in the aorta and its surrounds.

Another animal model, the Tg53 rat (Mol. Med. 7:831-44 (2001)), will be studied to demonstrate treatment and management of coronary artery disease, including plaque erosion, using the composition and methods of the present invention. This model will also be used to study other plaque-related phenomenon such as plaque inflammation, matrix degradation, apoptosis, neovascularization, thrombosis and hemorrhage, recapitulating the features and heterogeneity of human plaque disease. Plaque-laden animals will be identified by angiography, thermography, intravascular ultrasound, and/or a probe to measure proteolytic activity. Two groups of animals will be maintained similarly, except one group will receive an effective amount of implantable material. Reduction of and/or amelioration of plaque erosion and other indicia of coronary artery disease will be monitored over time. It is expected that rats treated with the materials and methods of the present invention will display reduction and/or amelioration of plaque erosion in the coronary arteries and coronary artery disease.

### 2. Plaque Fissure

The Tg53 rat (Mol. Med. 7:831-44 (2001)) will be studied to demonstrate treatment and management of coronary artery disease, including plaque fissure, using the composition and methods of the present invention. This model will also be used to study other plaque-related phenomenon such as plaque inflammation, matrix degradation, apoptosis, neovascularization, thrombosis and hemorrhage, recapitulating the features and heterogeneity of human plaque disease. Plaque-laden animals will be identified by angiography, thermography, intravascular ultrasound, and/or a probe to measure proteolytic activity. Two groups of animals will be maintained similarly, except one group will receive an effective amount of implantable material. Reduction of and/or amelioration of plaque fissure and other indicia of coronary artery disease will be monitored over time. It is expected that rats treated with the materials and methods of the present invention will display reduction and/or amelioration of plaque fissure in the coronary arteries and coronary artery disease.

Another model for studying plaque disease, including plaque fissure, is the FHC, hyperLDL-emic pig (Ann. NY Acad. Sci. 748:283-92 (1995)). This model can also be used to study the progression of coronary artery disease, including myocardial infarction. This model will be studied to demonstrate treatment and management of plaque disease, including plaque fissure and coronary artery disease, using the composition and methods of the present invention. Plaque-laden animals will be identified by angiography, thermography, intravascular ultrasound, and/or a probe to measure proteolytic activity. Two groups of animals will be maintained similarly, except one group will receive an effective amount of implantable material. Reduction of and/or amelioration of plaque fissure and disease progression will be monitored over time. It is expected that pigs treated with the materials and methods of the present invention will display reduction and/or amelioration of plaque disease, including plaque fissure and reduced incidence of coronary artery disease indicia including necrotic core lesions, fibrous caps, calcification, neovascularization, hemorrhage and fissuring.

### 3. Plaque Hemorrhage

The Tg53 rat (Mol. Med. 7:831-44 (2001)) will be studied to demonstrate treatment and management of coronary artery disease, including plaque hemorrhage, using the composition and methods of the present invention. This model will also be used to study other plaque-related phenomenon such as plaque inflammation, matrix degradation, apoptosis, neovascularization, thrombosis and hemorrhage, recapitulating the features and heterogeneity of human plaque disease. Plaque-laden animals will be identified by angiography, thermography, intravascular ultrasound, and/or a probe to measure proteolytic activity. Two groups of animals will be maintained similarly, except one group will receive an effective amount of implantable material. Reduction of and/or amelioration of plaque hemorrhage and other indicia of coronary artery disease will be monitored over time. It is expected that rats treated with the materials and methods of the present invention will display reduction and/or amelioration of plaque hemorrhage in the coronary arteries and coronary artery disease.

Other models for studying plaque disease, including plaque hemorrhage, is the FHC, hyperLDL-emic pig (Ann. N Y Acad. Sci. 748:283-92 (1995)). This model can also be used to study the progression of coronary artery disease, including myocardial infarction. This model will be studied to demonstrate treatment and management of coronary artery disease, including plaque disease and plaque fissure, using the composition and methods of the present invention. Plaque-laden animals will be identified by angiography, thermography, intravascular ultrasound, and/or a probe to measure proteolytic activity. Two groups of animals will be maintained similarly, except one group will receive an effective amount of implantable material. Reduction of and/or amelioration of plaque hemorrhage and disease progression will be monitored over time. It is expected that pigs treated with the materials and methods of the present invention will display reduction and/or amelioration of plaque disease, including plaque hemorrhage and a reduced incidence of coronary artery disease indicia including necrotic core lesions, fibrous caps, calcifications, neovascularization, hemorrhage and fissuring.

Another model for studying plaque hemorrhage as well as coronary artery atherosclerosis (CAA) is the African green monkey (Arterioscler. Thromb. 12:1274-83 (1992)). Animals fed diets rich in fat can be studied to demonstrate treatment and management of plaque hemorrhage as well as the etiology of CAA using the composition and methods of the present invention. Plaque-laden animals will be identified by angiography, thermography, intravascular ultrasound, and/or a probe to measure proteolytic activity. Two groups of animals will be maintained similarly, except one group will receive an effective amount of implantable material. Reduction of and/or amelioration of plaque hemorrhage as well as progression of CAA will be monitored over time. It is expected that monkeys treated with the materials and methods of the present invention will display reduction and/or amelioration of plaque hemorrhage as well as reduced incidence of CAA.

### 4. Plaque-Associated Occlusion

The Tg53 rat (Mol. Med. 7:831-44 (2001)) will be studied to demonstrate treatment and management of coronary artery disease, including plaque-associated occlusion, using the composition and methods of the present invention. This model will also be used to study other plaque-related phenomenon such as plaque inflammation, matrix degradation, apoptosis, neovascularization, thrombosis and hemorrhage, recapitulating the features and heterogeneity of human plaque disease. Plaque-laden animals will be identified by angiography, thermography, intravascular ultrasound, and/or a probe to measure proteolytic activity. Two groups of animals will be maintained similarly, except one group will receive an effective amount of implantable material. Reduction of and/or amelioration of plaque-associated occlusion and other indicia of coronary artery disease will be monitored over time. It is expected that rats treated with the materials and methods of the present invention will display reduction and/or amelioration of plaque-associate occlusion in the coronary arteries and coronary artery disease.

The pigeon model known as White Carneau (Arterioscler. Thromb. Vasc. Biol. 23:535-42 (2003); J. Hered. 92:439-42 (2001); Atherosclerosis 65:29-35 (1987); Arch. Pathol. Lab. Med. 102:581-6 (1978)) will be studied to demonstrate treatment and management of plaque disease, including plaque-associated occlusion, using the composition and methods of the present invention. Spontaneous plaque-laden animals will be identified by standard techniques such as angiography, thermography, intravascular ultrasound, and/or a probe to measure proteolytic activity. Two groups of animals will be maintained similarly, except one group will receive an effective amount of implantable material. Reduction of and/or amelioration of plaque disease, including plaque-associated occlusion, will be monitored over time. It is expected that pigeons treated with the materials and methods of the present invention will display reduction and/or amelioration of at least plaque-associated occlusion in the aorta and its surrounds.

Another model for studying plaque disease, including plaque-associated occlusion, is the FHC, hyperLDL-emic pig (Ann. NY Acad. Sci. 748:283-92 (1995)). This model can also be used to study the progression of coronary artery disease, including myocardial infarction. This model will be studied to demonstrate treatment and management of coronary artery disease, including plaque-associated occlusion, using the composition and methods of the present invention. Plaque-laden animals will be identified by angiography, thermography, intravascular ultrasound, and/or a probe to measure proteolytic activity. Two groups of animals will be maintained similarly, except one group will receive an effective amount of implantable material. Reduction of and/or amelioration of plaque-associated occlusion and disease progression will be monitored over time. It is expected that pigs treated with the materials and methods of the present invention will display reduction and/or amelioration of plaque disease, including plaque-associated occlusion and a reduced incidence of coronary artery disease indicia including necrotic core lesions, fibrous caps, calcification, neovascularization, hemorrhage and fissuring.

An additional model for studying plaque-associated occlusion is the WatanabeHHL MI rabbit (J. Atheroscler. Thromb. 11:184-9 (2004); Circulation 97:2433-44 (1998)). This is also a model of spontaneous myocardial infarction which displays types of plaques correlated with sudden cardiac events. This model is typified by nearly occluded plaques caused by luminal macrophage accumulation. This model will be studied to demonstrate treatment and management of plaque-associated occlusion and incidence of myocardial infarction using the composition and methods of the present invention. Plaque-laden animals will be identified by angiography, thermography, intravascular ultrasound, and/or a probe to measure macrophage accumulation and/or proteolytic activity. Two groups of animals will be maintained similarly, except one group will receive an effective amount of implantable material. Reduction of and/or amelioration of plaque-associated occlusion and incidence of myocardial infarction will be monitored over time. It is expected that rabbits treated with the materials and methods of the present invention will display reduction and/or amelioration of plaque-associated occlusion as well as reduced incidence of myocardial infarction.

### 5. Plaque-Associated Thrombosis

Another model for studying plaque-associated thrombosis as well as plaque rupture is the ApoE/LDLr knockout mouse (Arterioscler. Thromb. Vasc. Biol. 23:1608-14 (2003); Arterioscler. Thromb. Vasc. Biol. 22:788-92 (2002); Circulation 105:2766-71 (2002)). When such mice are fed a fat rich diet, this model can be studied to demonstrate treatment and management of plaque-associated thrombosis and plaque rupture using the composition and methods of the present invention. Fat fed mice will develop plaque lesions that rupture and form luminal thromboses. Plaque-laden animals will be identified by angiography, thermography, intravascular ultrasound, and/or a probe to measure proteolytic activity. Two groups of animals will be maintained similarly, except one group will receive an effective amount of implantable material. Reduction of and/or amelioration of plaque-associated thrombosis and rupture will be monitored over time. It is expected that mice treated with the materials and methods of the present invention will display reduction and/or amelioration of plaque-associated thrombosis and plaque rupture.

The Tg53 rat (Mol. Med. 7:831-44 (2001)) will be studied to demonstrate treatment and management of coronary artery disease, including plaque-associated thrombosis using the composition and methods of the present invention. This model will also be used to study other plaque-related phenomenon such as plaque inflammation, matrix degradation, apoptosis, neovascularization, thrombosis and hemorrhage, recapitulating the features and heterogeneity of human plaque disease. Plaque-laden animals will be identified by angiography, thermography, intravascular ultrasound, and/or a probe to measure proteolytic activity. Two groups of animals will be maintained similarly, except one group will receive an effective amount of implantable material. Reduction of and/or amelioration of plaque-associated thrombosis and other indicia of coronary artery disease will be monitored over time. It is expected that rats treated with the materials and methods of the present invention will display reduction and/or amelioration of plaque-associated thrombosis erosion in the coronary arteries and coronary artery disease.

### 6. Diet-Induced Hypercholesterolemic Animal Models

White New Zealand rabbits (Circulation 97:2433-44 (1998)) will be used in an induced model of atherosclerosis. Susceptibility to development of plaque lesions or plaque-like lesions will be induced via a diet of 0.2% cholesterol for 4 weeks. Susceptible animals will be identified by angiography, thermography, intravascular ultrasound, and/or a probe to measure proteolytic activity. Animals will then be subjected to an induced injury using a balloon catheter in a bilateral iliac artery denudation procedure. Accumulation of plaque lesions or plaque-like lesions will be monitored thereafter. Two groups of animals will be maintained similarly, except one group will receive an effective amount of implantable material. Reduction of and/or amelioration of plaque disease will be monitored over time. It is expected that rabbits treated with the materials and methods of the present invention will display reduction and/or amelioration of plaque disease.

### 7. Human Study

A population of plaque-laden candidates not yet experiencing ACS will be identified using, for example but not limited to, markers associated with vulnerable patients, as that term is defined above. For example, candidates will be identified for vulnerable myocardium, for example, by taking a medical history. Candidates will also be identified for the presence of vulnerable blood markers present in their serum, including but not limited to C-reactive protein, interleukin-6, and/or adhesion molecules. Vulnerable plaque will also be identified in candidates using, for example, angiography, thermography, intravascular ultrasound, and/or NIR spectroscopy to measure proteolytic activity.

The population will be divided into two groups, one of which will receive an effective amount of implantable material of the present invention. Reduction of and/or amelioration of the extent and severity of plaque disease will be monitored over time using angiography, thermography, intravascular ultrasound, and/or a probe to measure proteolytic activity. Also, the groups will be compared for incidence of ACS. It is expected that candidates treated with the materials and methods of the instant invention will display a reduction and/or amelioration of plaque disease, and treated candidates will exhibit a lower incidence of ACS.

A population of plaque-laden candidates having had at least one episode of ACS will also be identified. The population will be divided into two groups, one of which will receive an effective amount of implantable material of the present invention. Reduction of and/or amelioration of the extent and severity of plaque disease will be monitored over time using, for example but not limited to, angiography, thermography, intravascular ultrasound, and/or a probe to measure proteolytic activity. Also, the groups will be compared for incidence of clinical sequelae of ACS, for example myocardial infarction. It is expected that candidates treated with the materials and methods of the instant invention will display a reduction and/or amelioration of plaque disease, and treated candidates will exhibit a lower incidence of sequelae such as myocardial infarction.

The invention may be embodied in other specific forms without departing from the spirit or essential characteristics thereof. The present embodiments are therefore to be considered illustrative and not restrictive, the scope of the invention being indicated by the appended claims rather than by the foregoing description, and all changes which come within the meaning and range of equivalency of the claims are therefore intended to be embraced therein.

1-29. (canceled)

30. A method of treating a plaque-burdened site, the method comprising the step of:
contacting with an implantable material an exterior surface of a blood vessel at or adjacent or in the vicinity of a plaque-burdened site on an interior lumen of said vessel, wherein said implantable material comprises cells attached via cell to matrix interactions to a biocompatible matrix; wherein said implantable material is in an amount effective to treat the plaque-burdened site; and wherein the effective amount comprises at least about 2×10⁵ to 4×10⁵ cells/cm³ implantable material, wherein said cells are at least about 80% viable, wherein said cells produce at least about 0.5 to 1.0 micrograms heparan sulfate/10⁶ cells/day, wherein said cells produce at least about 200 to 300 picograms TGF-β₁/ml/day, and wherein said cells produce no more than about 200 to 400 picograms b-FGF/ml/day.

31. The method of claim 30 wherein said effective amount reduces plaque hemorrhage at the plaque-burdened site.

32. The method of claim 30 wherein said effective amount reduces plaque fissure at the plaque-burdened site.

33. The method of claim 30 wherein said effective amount reduces plaque erosion at the plaque-burdened site.

34. The method of claim 30 wherein said effective amount reduces rupture, displacement or dislodgement of plaque at the plaque-burdened site.

35. A method of diminishing clinical sequelae associated with vulnerable plaque in a patient in need thereof, the method comprising the step of:
contacting with an implantable material an exterior surface of said blood vessel at or adjacent or in the vicinity of a plaque-burdened site on the interior lumen of said vessel, wherein said implantable material comprises cells attached via cell to matrix interactions to a biocompatible matrix; wherein said implantable material is in an amount effective to diminish clinical sequelae associated with vulnerable plaque, said clinical sequelae selected from the group consisting of: acute coronary syndrome, myocardial infarction, sudden cardiac death; and wherein the amount effective comprises a total cell load per patient no less than about 2×10³ to no more than about 2×10⁶ cells per kilogram body weight.

36. The method of claim 30 or claim 35, wherein deposition of the implantable material is accomplished by first traversing or penetrating an interior wall of said blood vessel and then depositing the implantable material on the exterior surface of said blood vessel at or adjacent or in the vicinity of the plaque-burdened site.

37. The method of claim 30 or claim 35, wherein deposition of the implantable material is accomplished by entering the perivascular space by percutaneous administration and then depositing the implantable material at or adjacent or in the vicinity of the plaque-burdened site.

38. An effective amount of an implantable material suitable for use with the method of claim 35, wherein the effective amount comprises at least about 2×10⁵ to 4×10⁵ cells/cm³ implantable material, wherein said cells are at least about 80% viable, wherein said cells produce at least about 0.5 to 1.0 micrograms heparan sulfate/10⁶ cells/day, wherein said cells produce at least about 200 to 300 picograms TGF-β₁/ml/day, and wherein said cells produce no more than about 200 to 400 picograms b-FGF/ml/day.

39. The implantable material of claim 38 wherein the implantable material is a flexible planar form.

40. The implantable material of claim 38 wherein the implantable material is a flowable composition.

41. The implantable material of claim 38 wherein said cells are endothelial cells or cells having an endothelial-like phenotype.

42. The implantable material of claim 41 wherein said cells are selected from the group consisting of: a confluent population of cells; a near confluent population of cells; a post confluent population of cells; a non-exponential population of cells; and cells which have a phenotype of any one of the foregoing population of cells.

43. The method of claim 30 or claim 35, wherein the exterior surface of said blood vessel is a non-luminal surface selected from the group consisting of: perivascular space, an adventitial site, a medial site, an intimal site, and a combination of any one of the foregoing.

44. The method of claim 30 wherein the total cell load per patient comprises about 1.6×10⁴ to 2.6×10⁴ cells per kilogram body weight.

45. The method of claim 30 or claim 35 wherein the contacting step is accomplished by depositing the implantable material on the exterior surface of said blood vessel either proximal or distal to a plaque-burdened site.

46. The method of claim 45 wherein the contacting step is accomplished by depositing the implantable material within about 2 to 20 mm of the plaque-burdened site.

47. The method of claim 45 wherein the contacting step is accomplished by depositing the implantable material within about 21 to 40 mm of the plaque-burdened site.

48. The method of claim 45 wherein the contacting step is accomplished by depositing the implantable material within about 41 to 60 mm of the plaque-burdened site.

49. The method of claim 45 wherein the contacting step is accomplished by depositing the implantable material within about 61 to 100 mm of the plaque-burdened site.
